(19) United States
(12) Patent Application Publication
(10) Pub. No.: US 2003/0007974 A1
(43) Pub. Date: Jan. 9, 2003

(54) Endopeptidase/anti-PSMA antibody fusion proteins for treatment of cancer

(76) Inventors: David M. Nanus, New Rochelle, NY (US); Shen Ruoqian, Corona, NY (US); Neil Bander, New York, NY (US)

Correspondence Address: LAURIE BUTLER LAWRENCE, Fish & Richardson P.C., 225 Franklin Street, Boston, MA, 02110-2804, US

(21) Appl. No.: 10/160,994

(22) Filed: May 30, 2002

Related U.S. Application Data
(60) Provisional application No. 60/294,359, filed on May 30, 2001.

(51) Int. Cl. A61K 39/395; C12N 9/00

(52) U.S. Cl. 424/178.1; 435/188.5

(57) ABSTRACT

A fusion protein for treatment of prostate cancer and other cancers which includes an endopeptidase bound to a monoclonal antibody, or an antigen binding portion thereof, specific for prostate specific membrane antigen (PSMA)expressed either on the tumor cell surface or on the surface of epithelial cells within the tumor. The invention also features pharmaceutical compositions comprising such fusion proteins as well as methods of manufacture and treatment with such fusion proteins.

This application claims priority to U.S. provisional application No. 60/294,359 filed on May 30, 2001, the contents of which is incorporated herein by reference.

### BACKGROUND

Prostate cancer is one of the most common causes of cancer deaths in American males. In 1999, approximately 185,000 new cases were diagnosed and 37,500 died of this disease (NCI SEER data). It accounts for about 40% of all cancers diagnosed in men. A male born in the U.S. in 1990 has approximately a 1 in 8 likelihood of being diagnosed with clinically apparent prostate cancer in his lifetime. Even prior to the recent increase in incidence, prostate cancer was the most prevalent cancer in men (Feldman, A. R. et al. (1986) NEJM315:1394-7). There is currently very limited treatment for prostate cancer once it has metastasized (spread beyond the prostate). Currently, systemic therapy is limited to various forms of androgen (male hormone) deprivation. While most patients will demonstrate initial clinical improvement, virtually inevitably, androgen-independent cells develop. Endocrine therapy is thus palliative, not curative. In a study of 1387 patients with metastatic disease detectable by imaging (e.g., bone or CT scan), the median time to objective disease progression (excluding biochemical/PSA progression) after initiation of hormonal therapy (i.e., development of androgen-independence) was 16-48 months (Eisenberger M. A., et al. (1998) NEJM 339:103642). Median overall survival in these patients was 28-52 months from the onset of hormonal treatment (Eisenberger M. A., et al. (1998) supra.). Subsequent to developing androgen-independence, there is no effective standard therapy and the median duration of survival is 9-12 months (Vollmer, R. T., et al. (1999) Clin Can Res 5: 831-7; Hudes G., et al., (1997) Proc Am Soc Clin Oncol 16:316a (abstract); Pienta K. J., et al. (1994) J Clin Oncol 12(10):2005-12; Pienta K. J., et al. (1997) Urology 50:401-7; Tannock I. F., et al., (1996) J Clin Oncol 14:1756-65; Kantoff P. W., et al., (1996) J Clin. Oncol. 15 (Suppl):25:110-25). Cytotoxic chemotherapy is poorly tolerated in this age group and generally considered ineffective and/or impractical. In addition, prostate cancer is relatively resistant to cytotoxic agents. Thus, chemotherapeutic regimen has not demonstrated a significant survival benefit in this patient group.

For men with a life expectancy of less than 10 years, watchful waiting is appropriate where low-grade, low-stage prostate cancer is discovered at the time of a partial prostatectomy for benign hyperplasia (W. J. Catalona, (1994) New Engl. J Med. 331(15):996-1004). Such cancers rarely progress during the first five years after detection. On the other hand, for younger men, curative treatment is often more appropriate.

Where prostate cancer is localized and the patient's life expectancy is 10 years or more, radical prostatectomy offers the best chance for eradication of the disease. Historically, the drawback of this procedure is that most cancers had spread beyond the bounds of the operation by the time they were detected. However, the use of prostate-specific antigen testing has permitted early detection of prostate cancer. As a result, surgery is less extensive with fewer complications. Patients with bulky, high-grade tumors are less likely to be successfully treated by radical prostatectomy.

After surgery, if there are detectable serum prostate-specific antigen concentrations, persistent cancer is indicated. In many cases, prostate-specific antigen concentrations can be reduced by radiation treatment. However, this concentration often increases again within two years.

Radiation therapy has also been widely used as an alternative to radical prostatectomy. Patients generally treated by radiation therapy are those who are older and less healthy and those with higher-grade, more clinically advanced tumors. Particularly preferred procedures are external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

For treatment of patients with locally advanced disease, hormonal therapy before or following radical prostatectomy or radiation therapy has been utilized. Hormonal therapy is the main form of treating men with disseminated prostate cancer. Orchiectomy reduces serum testosterone concentrations, while estrogen treatment is similarly beneficial. Diethylstilbestrol from estrogen is another useful hormonal therapy which has a disadvantage of causing cardiovascular toxicity. When either LHRH agonists, such as leuprolide, buserelin, or goserelin, or gonadotropin-releasing hormone antagonists, such as Abarelix, are administered testosterone concentrations are ultimately reduced. Flutamide and other nonsteroidal, anti-androgen agents block binding of testosterone to its intracellular receptors. As a result, it blocks the effect of testosterone, increasing serum testosterone concentrations and allows patients to remain potent a significant problem after radical prostatectomy and radiation treatments.

In view of the shortcoming of existing therapies, there exists a need for improved modalities for preventing and treating cancers, such as prostate cancer.

### SUMMARY

The invention is based, in part, on the discovery that fusion proteins which include an endopeptidase and an antibody (or portion thereof) specific for prostate specific membrane antigen (PSMA) can be used to increase the concentration of endopeptidase delivered to PSMA expressing cells. Various peptide growth factors have been shown to contribute to the growth and development of hormone refractory prostate cancer. These include atrial natriuretic factor, substance P, bamdykinin, oxytocin, Leu- and Met-enkephalins, neurotensin, bombesin, endothelin-1 and bombesin-like peptides. Endopeptidases such neural endopeptidase 24.11 (NEP) inactivate these peptide factors by, for example, cleaving peptide bonds within the peptides. It has been found that expression of endopeptidases such as NEP is decreased in patients having hormone-refractory prostate cancers, suggesting that the loss of endopeptidase expression contributes to the development of peptide growth factors ability to stimulate prostate cancer growth and progression to becoming hormone refractory. By using anti-PSMA antibodies or fragments thereof as part of a fusion protein with an endopeptidase, localized delivery of the endopeptidase at sufficient concentrations to cancerous cells can be achieved.

Accordingly, in one aspect, the invention features fusion protein which includes an endopeptidase bound to an antibody, or an antigen binding portion thereof, specific for PSMA. The enodpeptidase can be a neutral endopeptidase, or another peptidase which recognizes and cleaves one or more protein factors required for growth and/or progression of a tumor. Examples of such protein factors which the enodpeptidase can recognize and cleave include atrial natriuretic factor, substance P, bradykinin, oxytocin, Leuenkephalins, Met-enkephalins, neurotensin, bombesin, endothelin-1, and bombesin-like peptides.

In a preferred embodiment, the endopeptidase is neutral endopeptidase 24.11 (NEP).

In another embodiment, the anti-PSMA antibody can be a monoclonal or polyclonal antibody or antigen binding fragment thereof. Preferably, the anti-PSMA antibody is a monoclonal antibody or antigen binding portion thereof. The anti-PSMA antibodies (e.g., recombinant or modified antibodies) can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)₂ or scFv fragment, or one or more CDRs). An antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-PSMA antibody includes a heavy and light chain constant region substantially from a human antibody, e.g., a human IgG1 constant region or a portion thereof.

The antibody (or fragment thereof) portion of the fusion protein can be a murine or a human antibody. Examples of preferred murine monoclonal antibodies that can be used include a E99, J415, J533 and J591 antibody, which are produced by hybridoma cell lines having an ATCC Accession Number HB-12101, HB-12109, HB-12127, and HB-12126, respectively.

Also within the scope of the invention are fusion proteins which have an antibody, or antigen-binding fragments thereof, portion which binds to an overlapping epitope of, or competitively inhibits, the binding of the anti-PSMA antibodies disclosed herein to PSMA, e.g., an antibody which binds an overlapping epitope of, or competitively inhibits, the binding of monoclonal antibodies E99, J415, J533 or J591 to PSMA.

In other embodiments, the antibody (or fragments thereof) portion of the fusion protein can be a recombinant or modified anti-PSMA antibody chosen from, e.g., a chimeric, a humanized, a deimmunized, or an in vitro generated antibody. The modified antibodies can be CDR-grafted, humanized, deimmunized, or more generally, antibodies having CDRs from a non-human antibody, e.g., murine J591, J415, J533 or E99 antibody and a framework that is selected as less immunogenetic in humans, e.g., less antigenic than the murine framework in which a murine CDR naturally occurs.

In other embodiments, the antibody portion of the fusion protein interacts with, e.g., binds to, PSMA, preferably human PSMA, with high affinity and specificity. For example, the antibody portion binds to human PSMA with an affinity constant of at least 10⁷M⁻¹, preferably between 10⁸ M⁻¹ and 10¹⁰ M⁻¹, or about 10⁹ M⁻¹. Preferably, the antibody portion interacts with, e.g., binds to, the extracellular domain of PSMA, and most preferably, the extracellular domain of human PSMA (e.g., amino acids 44-750 of human PSMA).

In another embodiment, the fusion protein further includes a linker or hinge region, e.g. a linker or hinge region between the endopeptidase portion and the antibody portion thereof.

In another aspect, the invention features a pharmaceutical composition which includes a fusion protein described herein and a pharmaceutically acceptable carrier, excipient, or stabilizer.

In another embodiment, the invention features a nucleic acid encoding a fusion protein described herein. The invention also features vectors containing such sequences as well as host cells into which the nucleic acid sequence has been introduced.

Methods of using the fusion proteins of the invention to deliver an endopeptidase to a PSMA expressing cell, e.g., a PSMA expressing cancer, a prostatic or a vascular cell, either in vivo or in vitro, are also encompassed by the invention. Accordingly, in another aspect, the invention features methods of treating or preventing a disorder, e.g., a prostatic disorder (e.g., a cancerous or non-cancerous disorder, e.g., a benign or hyperplastic prostatic disorder) or a nonprostatic disorder (e.g., a cancer, e.g., a malignant cancer) by administering to a subject a fusion protein described herein, in an amount effective to treat or prevent such disorder. Examples of prostatic disorders that can be treated or prevented include, but are not limited to, genitourinary inflammation (e.g., inflammation of smooth muscle cells) as in prostatitis; benign enlargement, for example, nodular hyperplasia (benign prostatic hypertrophy or hyperplasia); and cancer, e.g., adenocarcinoma or carcinoma, of the prostate and/or testicular tumors. Methods and compositions disclosed herein are particularly useful for treating metastatic lesions associated with prostate cancer, e.g., hormone refractory prostate cancer. In some embodiments, the patient will have undergone one or more of prostatectomy, chemotherapy, or other anti-tumor therapy and the primary or sole target will be metastatic lesions, e.g., metastases in the bone marrow or lymph nodes. Examples of non-prostatic cancerous disorders include, but are not limited to, solid tumors, soft tissue tumors, and particularly metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), genitals and genitourinary tract (e.g., renal, urothelial, bladder cells), pharynx, CNS (e.g., neural or glial cells), skin (e.g., melanoma), and pancreas, as well as adenocarcinomas which include malignancies such as most colon cancers, rectal cancer, renal-cell carcinoma, liver cancer, non-small cell carcinoma of the lung, cancer of the small intestine and cancer of the esophagus. In some embodiments, the subject will have undergone one or more of surgical removal of a tissue, chemotherapy, or other anti-cancer therapy and the primary or sole target will be metastatic lesions, e.g., metastases in the bone marrow or lymph nodes.

In a preferred embodiment the subject is treated to prevent a disorder, e.g., a prostatic disorder, e.g., hormone refractory prostate cancer. The subject can be one at risk for the disorder, e.g., a subject having a relative afflicted with the disorder, e.g., a subject with one or more of a grandparent, parent, uncle or aunt, sibling, or child who has or had the disorder, or a subject having a genetic trait associated with risk for the disorder. In a preferred embodiment the disorder is a prostatic disorder (e.g., a cancerous or non-cancerous prostatic disorder, e.g., a benign or hyperplastic prostatic disorder), or a non-prostatic disorder (e.g., cancer, e.g., malignant cancer) and the subject has one or more of a grandfather, father, uncle, brother, or son who has or had the disorder, or a subject having a genetic trait associated with risk for the disorder.

The subject can be a mammal, e.g., a primate, preferably a higher primate, e.g., a human (e.g., a patient having, or at risk of, a disorder described herein, e.g., a prostatic or a cancer disorder). In one embodiment, the subject is a patient having prostate cancer (e.g., a patient suffering from recurrent, hormone refractory or metastatic prostate cancer).

The endopeptidase/anti-PSMA antibody fusion protein, e.g., the fusion proteins described herein, can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing the effect of recombinant NEP (rNEP) on growth of the androgen-independent prostate cancer cell lines PC-3 and TSU-Pr1.

FIG. 2 is a Western blot showing NEP expression and FAK phosphorylation and cell migration in prostate cancer cells.

FIG. 3 is a graph showing cell migration in prostate cancer cells.

FIG. 4 is a graph showing cell proliferation in WT-5 cells, which express high levels of enzymatically active NEP protein when cultured in the absence of tetracycline.

FIG. 5 is a magnetic resonance image of WT-5 and TN12 cells injected directly into the prostate gland of athymic mice.

FIG. 6 is a Western blot of cell lysates from CHO cells infected with JNEP or Pz1. The blot was probed with anti-NEP monoclonal antibody.

FIG. 7A is a graph of PC-3/PSMA and PC-3/FLU cell growth inhibition by an NEP/anti-PSMA antibody fusion protein

FIG. 7B is a graph showing the concentration dependence of cell growth inhibition by a NEP/anti-PSMA antibody fusion protein.

### DETAILED DESCRIPTION

The present invention provides a fusion protein for treatment of cancerous tumors that include cells that express PSMA on an extracellular surface. Human PSMA is expressed on the surface of normal, benign hyperplastic, and cancerous prostate epithelial cells, as well as vascular endothelial cells proximate to cancerous cells, e.g., renal, urothelial (e.g., bladder), testicular, colon, rectal, lung (e.g., non-small cell lung carcinoma), breast, liver, neural (e.g., neuroendocrine), glial (e.g., glioblastoma), pancreatic (e.g., pancreatic duct), melanoma (e.g., malignant melanoma), or soft tissue sarcoma cancerous cells. The expression of human PSMA is substantially lower on non-malignant prostate cells where PSM′, a splice variant that lacks a portion of the N-terminal domain that includes the transmembrane domain, is more abundant. Due to the absence of the N-terminal region containing the transmembrane domain, PSM′ is primarily cytoplasmic and is not located on the cell membrane.

The fusion protein includes at least a) an endopeptidase effective to recognize, and cleave growth factors required for growth and/or progression of the cancerous tumor, linked to b) a monoclonal antibody, or an antigen binding portion thereof, that binds to PSMA. The fusion protein may also include additional portions, such as a hinge or linker region. Hinge and linker sequences are known in the art.

Preferably, the endopeptidase activity does not significantly interfere with growth and/or viability of noncancerous cells. Examples of such protein factors which the endopeptidase can recognize and cleave include atrial natriuretic factor, substance P, bradykinin, oxytocin, Leu-enkephalins, Met-enkephalins, neurotensin, bombesin, endothelin-1, and bombesin-like peptides. Preferably, the endopeptidase is neutral endopeptidase 24.11 (also referred to as NEP, neprilysin, enkephalinase, CD10 and EC 3.4.24.11). Neutral endopeptidase 24.11 is a 90 to 110 kDa zinc dependent cell surface metallopeptidase which cleaves peptide bonds on the amino side of hydrophobic amino acids. NEP is a type II integral membrane protein, containing an inverted membrane orientation and possessing an extracellular carboxyl terminus which contains an active catalytic domain. NEP inactivates a variety of peptides, including atrial natriuretic factor, substance P, bradykinin, oxytocin, Leu-enkephalins, Met-enkephalins, neurotensin, bombesin, endothelin-1, and bombesin-like peptides. The endopeptidase, e.g., NEP, portion of the fusion protein reduces the local concentration of peptide available for receptor binding and signal transduction. The term “reduces” as used herein refers to decreasing the concentration of a peptide as compared to the concentration in the absence of the fusion protein.

The monoclonal antibody, or an antigen binding portion thereof, preferably binds to an extracellular domain of PSMA, that is, a portion of PSMA that is located on the surface of the PSMA-expressing cell. As used herein, “PSMA” or “prostate-specific membrane antigen” protein refers to mammalian PSMA, preferably human PSMA protein. Human PSMA includes the two protein products, PSMA and PSM′, encoded by the two alternatively spliced mRNA variants (containing about 2,653 and 2,387 nucleotides, respectively) of the PSMA cDNA disclosed in Israeli et al. (1993) Cancer Res. 53:227-230; Su et al. (1995) Cancer Res. 55:1441-1443; U.S. Pat. Nos. 5,538,866, US 5,935,818, and WO 97/35616, the contents of which are hereby incorporated by reference. The long transcript of PSMA encodes a protein product of about 100-120 kDa molecular weight characterized as a type II transmembrane receptor having sequence identity with the transferrin receptor and having NAALADase activity (Carter et al. (1996) Proc. Natl. Acad. Sci. USA 93:749-753). Accordingly, the term “human PSMA” refers to at least two protein products, human PSMA and PSM′, which have or are homologous to (e.g., at least about 85%, 90%, 95% identical to) an amino acid sequence as shown in Israeli et al. (1993) Cancer Res. 53:227-230; Su et al. (1995) Cancer Res. 55:1441-1443; U.S. Pat. Nos. 5,538,866, US 5,935,818, and WO 97/35616; or which is encoded by (a) a naturally occurring human PSMA nucleic acid sequence (e.g., Israeli et al. (1993) Cancer Res. 53:227-230 or U.S. Pat. No. 5,538,866); (b) a nucleic acid sequence degenerate to a naturally occurring human PSMA sequence; (c) a nucleic acid sequence homologous to (e.g., at least about 85%, 90%, 95% identical to) the naturally occurring human PSMA nucleic acid sequence; or (d) a nucleic acid sequence that hybridizes to one of the foregoing nucleic acid sequences under stringent conditions, e.g., highly stringent conditions.

An “anti-PSMA antibody” is an antibody that interacts with (e.g., binds to) PSMA, preferably human PSMA protein. Preferably, the anti-PSMA antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866). In one embodiment, the anti-PSMA antibody binds all or part of the epitope of an antibody described herein, e.g., J591, E99, J415, and J533. The anti-PSMA antibody can inhibit, e.g., competitively inhibit, the binding of an antibody described herein, e.g., J591, E99, J415, and J533, to human PSMA. An anti-PSMA antibody may bind to an epitope, e.g., a conformational or a linear epitope, which epitope when bound prevents binding of an antibody described herein, J591, E99, J415, and J533. The epitope can be in close proximity spatially or functionally-associated, e.g., an overlapping or adjacent epitope in linear sequence or conformationally to the one recognized by the J591, E99, J415, or J533 antibody. In one embodiment, the anti-PSMA antibody binds to an epitope located wholly or partially within the region of about amino acids 120 to 500, preferably 130 to 450, more preferably, 134 to 437, or 153 to 347, of human PSMA (amino acid residues correspond to the human PSMA sequence disclosed in U.S. Pat. No. 5,538,866). Preferably, the epitope includes at least one glycosylation site, e.g., at least one N-linked glycosylation site (e.g., the N-linked glycosylation site located at about amino acids 190-200, preferably at about amino acid 195, of human PSMA) (amino acid residues correspond to the human PSMA sequence disclosed in U.S. Pat. No. 5,538,866).

In a preferred embodiment, the interaction, e.g., binding, between an anti-PSMA antibody and PSMA occurs with high affinity (e.g., affinity constant of at least 10⁷ M⁻¹, preferably, between 10⁸ M⁻¹ and 10^{10,} or about 10⁹ M⁻¹) and specificity. Examples of anti-PSMA antibodies include, e.g., monospecific, monoclonal (e.g., human), recombinant or modified, e.g., chimeric, CDR-grafted, humanized, deimmunized, and in vitro generated anti-PSMA antibodies.

The fusion protein is contacted with the tumor cells under conditions effective to permit binding of the fusion protein to the extracellular domain of PSMA and also effective to permit cleavage of the growth factors required for growth and/or progression of the cancerous tumor. The contacting is preferably performed in vivo, in a living mammal, such as a human.

As used herein, the term “treat” or “treatment” is defined as the application or administration of a endopeptidase/anti-PSMA antibody fusion protein to a subject, e.g., a patient, or application or administration to an isolated tissue or cell from a subject, e.g., a patient, which is returned to the patient. The binding agent can be administered alone or in combination with, a second agent. The subject can be a patient having a disorder (e.g., a disorder as described herein), a symptom of a disorder or a predisposition toward a disorder. The treatment can be to cure, heal, alleviate, relieve, alter, remedy, ameliorate, improve or affect the disorder, the symptoms of the disorder or the predisposition toward the disorder. While not wishing to be bound by theory, treating is believed to cause the inhibition, ablation, or killing of a cell in vitro or in vivo, or otherwise reducing capacity of a cell, e.g., an aberrant cell, to mediate a disorder, e.g., a disorder as described herein (e.g., prostate cancer).

As used herein, an amount of an endopeptidase/anti-PSMA antibody fusion protein, effective to treat a disorder, or a “therapeutically effective amount” refers to an amount of the fusion protein which is effective, upon single or multiple dose administration to a subject, in treating a cell, e.g., a skin cell (e.g., a PSMA-expressing skin cell, or a vascular cell proximate thereto), or in prolonging curing, alleviating, relieving or improving a subject with a disorder as described herein beyond that expected in the absence of such treatment. As used herein, “inhibiting the growth” of the lesion refers to slowing, interrupting, arresting or stopping its growth and does not necessarily indicate a total elimination of the growth or lesion.

As used herein, an amount of a endopeptidase/anti-PSMA fusion protein effective to prevent a disorder, or a “prophylactically effective amount” of the fusion protein refers to an amount of a fusion protein, e.g., a endopeptidase/anti-PSMA antibody fusion protein as described herein, which is effective, upon single- or multiple-dose administration to the subject, in preventing or delaying the occurrence of the onset or recurrence of a disorder, e.g., cancer, as described herein, or treating a symptom thereof.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the PSMA-expressing hyperproliferative cells” means that the rate of growth of the cells will be different, e.g., statistically significantly different, from the untreated cells.

As used herein, “specific binding” refers to the property of the anti-PSMA antibody, to: (1) to bind to PSMA, e.g., human PSMA protein, with an affinity of at least 1×10⁷ M⁻¹, and (2) preferentially bind to PSMA, e.g., human PSMA protein, with an affinity that is at least two-fold, 50-fold, 100-fold, 1000-fold, or more greater than its affinity for binding to a nonspecific antigen (e.g., BSA, casein) other than PSMA.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Preferably, each VH and VL is composed of three CDRs and four FRs, arranged from amino-terminus to carboxy-terminus in the following order: FRI, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or from a sequence generated by phage display, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., a mouse, framework modified to decrease antigenicity in humans, or a synthetic framework, e.g., a consensus sequence. These are sometimes referred to herein as modified immunoglobulins. A modified antibody, or antigen binding fragment thereof, includes at least one, two, three or four modified immunoglobulin chains, e.g., at least one or two modified immunoglobulin light and/or at least one or two modified heavy chains. In one embodiment, the modified antibody is a tetramer of two modified heavy immunoglobulin chains and two modified light immunoglobulin chains.

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to a portion of an antibody which specifically binds to PSMA (e.g., human PSMA), e.g., a molecule in which one or more immunoglobulin chains is not full length but which specifically binds to PSMA (e.g., human PSMA protein). Examples of binding fragments encompassed within the term “antigen-binding fragment” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated (e.g., by phage display) antibodies, and may optionally include constant regions derived from human germline immunoglobulin sequences.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Calculations of “homology” between two sequences can be performed as follows.

The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a homology limitation of the invention) are a Blossum 62 scoring matrix with a gap penalty of 12, a gap extend penalty of 4, and a frameshift gap penalty of 5.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0. 1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0. 1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified.

It is understood that one or more of the endopeptidase and/or anti-PSMA antibody (or antigen binding portion thereof) of the fusion protein may have additional conservative or non-essential amino acid substitutions, which do not have a substantial effect on the polypeptide functions. Whether or not a particular substitution will be tolerated, i.e., will not adversely affect desired biological properties, such as binding activity can be determined as described in Bowie, JU et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

Anti-PSMA Antibodies

Many types of anti-PSMA antibodies, or antigen-binding fragments thereof, are useful in the fusion proteins of this invention. The antibodies can be of the various isotypes, including: IgG (e.g., IgG1, IgG2, IgG3, IgG4), IgM, IgA1, IgA2, IgD, or IgE. Preferably, the antibody is an IgG isotype. The antibody molecules can be full-length (e.g., an IgG1 or IgG4 antibody) or can include only an antigen-binding fragment (e.g., a Fab, F(ab′)2, Fv or a single chain Fv fragment). These include monoclonal antibodies, recombinant antibodies, chimeric antibodies, humanized antibodies, deimmunized antibodies, as well as antigen-binding fragments of the foregoing.

As described in more detail below, antibodies (preferably, monoclonal antibodies from differing organisms, e.g., rodent, sheep, human) against a predetermined antigen can be produced using art-recognized methods. Once the antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions. A light and the heavy immunoglobulin chains can be generated as part of a vector for expressing the fusion protein for introduction into the appropriate host cells.

Monoclonal anti-PSMA antibodies can be used in the fusion proteins of the invention. Preferably, the monoclonal antibodies bind to the extracellular domain of PSMA (i.e., an epitope of PSMA located outside of a cell). Examples of preferred murine monoclonal antibodies to human PSMA include, but are not limited to, E99, J415, J533 and J591, which are produced by hybridoma cell lines having an ATCC Accession Number HB-12101, HB-12109, HB-12127, and HB-12126, respectively, all of which are disclosed in U.S. Pat. Nos. 6,107,090 and 6,136,311, the contents of which are expressly incorporated by reference.

Additional monoclonal antibodies to PSMA can be generated using techniques known in the art. Monoclonal antibodies can be produced by a variety of techniques, including conventional monoclonal antibody methodology e.g., the standard somatic cell hybridization technique of Kohler and Milstein, Nature 256: 495 (1975). See generally, Harlow, E. and Lane, D. (1988) Antibodies: A Laboratory Manual, Cold Spring Harbor Laboratory Press, Cold Spring Harbor, N.Y. Although somatic cell hybridization procedures are preferred, in principle, other techniques for producing monoclonal antibody can be employed e.g., viral or oncogenic transformation of B lymphocytes. The preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Useful immunogens for the purpose of this invention include PSMA (e.g., human PSMA)-bearing cells (e.g., dermal or epidermal cells from a subject with psoriasis or prostate tumor cell lines, e.g., LNCap cells); isolated or purified PSMA, e.g., human PSMA, e.g., biochemically isolated PSMA, or a portion thereof, e.g., the extracellular domain of PSMA. Techniques for generating antibodies to PSMA are described in U.S. Pat. Nos. 6,107,090, US 6,136,311, the contents of all of which are expressly incorporated by reference.

Human monoclonal antibodies (mAbs) directed against human proteins can be generated using transgenic mice carrying the human immunoglobulin genes rather than the mouse system. Splenocytes from these transgenic mice immunized with the antigen of interest are used to produce hybridomas that secrete human mabs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Anti-PSMA antibodies or fragments thereof useful in the present invention may also be recombinant antibodies produced by host cells transformed with DNA encoding immunoglobulin light and heavy chains of a desired antibody. Recombinant antibodies may be produced by known genetic engineering techniques. For example, recombinant antibodies may be produced by cloning a nucleotide sequence, e.g., a cDNA or genomic DNA sequence, encoding the immunoglobulin light and heavy chains of the desired antibody from a hybridoma cell that produces an antibody useful in this invention. The nucleotide sequence encoding those polypeptides is then inserted into an expression vector that also includes the sequence encoding the endopeptidase and, optionally a linker. Prokaryotic or eukaryotic host cells may be used.

It will be understood that variations in the procedure for producing the antibody as part of a fusion protein are useful in the present invention. For example, it may be desired to transform a host cell with DNA encoding either the light chain or the heavy chain (but not both) of an antibody. Recombinant DNA technology may also be used to remove some or all of the DNA encoding either or both of the light and heavy chains that is not necessary for PSMA binding, e.g., the constant region may be modified by, for example, deleting specific amino acids.

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl. Cancer Inst. 80:1553-1559).

An antibody or an immunoglobulin chain can be humanized by methods known in the art. Once the murine antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions.

As used herein, “an in vitro generated” “antibody” or “immunoglobulin” refers to an immunoglobulin where all or part of the variable region, e.g., one or more or all CDRs, is generated in a non-immune cell selection, e.g., an in vitro phage display, protein chip or any other method in which candidate sequences can be tested for their ability to bind to an antigen.

Anti-PSMA antibody that are not intact antibodies are also useful in this invention. Such antibodies may be derived from any of the antibodies described above. For example, antigen-binding fragments, as well as full-length monomeric, dimeric or trimeric polypeptides derived from the above-described antibodies are themselves useful. Useful antibody homologs of this type include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

Monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; (iii) by replacing the constant region with an endopeptidase or (iv) by modifying one or more amino acids in the constant region to alter, for example, the number of glycosylation sites, effector cell function, Fc receptor (FcR) binding, complement fixation, among others.

In one embodiment, the constant region of the antibody can be replaced by another constant region from, e.g., a different species. This replacement can be carried out using molecular biology techniques. For example, the nucleic acid encoding the VL or VH region of a antibody can be converted to a full-length light or heavy chain gene, respectively, by operatively linking the VH or VL-encoding nucleic acid to another nucleic acid encoding the light or heavy chain constant regions. The sequences of human light and heavy chain constant region genes are known in the art. Preferably, the constant region is human, but constant constant variable regions from other species, e.g., rodent (e.g., mouse or rat), primate, camel, rabbit, can also be used. Constant regions from these species are known in the art (see e.g., Kabat, E. A., et al. (1991) Sequences of Proteins of Immunological Interest, Fifth Edition, U.S. Department of Health and Human Services, NIH Publication No. 91-3242).

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 5 and 5,648,260, the contents of all of which are hereby incorporated by reference). Similar type of alterations could be described, which if applied to immunoglobulins of murine or other species, would reduce or eliminate these functions.

Pharmaceutical Compositions

In another aspect, the present invention provides compositions, e.g., pharmaceutically acceptable compositions, which include an endopeptidase/anti-PSMA antibody fusion protein described herein, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. The carrier is suitable for intravenous, intramuscular, subcutaneous, parenteral, spinal or epidermal administration (e.g., by injection or infusion). Depending on the route of administration, the active compound, i.e., the fusion protein may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the fusion protein is administered by intravenous infusion or injection (e.g., by needleless injection). In another preferred embodiment, the fusion protein is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., the fusion protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The fusion proteins can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In some embodiments, pharmaceutical compositions of fusion proteins, alone or in combination with other agent, can be delivered or administered topically or by transdermal patches for treating skin disorders. In those embodiments where the fusion protein is a small molecule, oral administration can be used. Additionally, the compositions can be delivered parenterally, and for direct injection of skin lesions. Parenteral therapy is typically intra-dermal, intra-articular, intramuscular or intravenous. Fusion proteins can be applied, in a cream or oil based carrier, directly to the psoriatic lesions. Alternatively, an aerosol can be used topically. These compounds can also be orally administered.

Intra-articular injection is a preferred alternative in the case of treating one or only a few (such as 2-6) joints. Additionally, the therapeutic compounds are injected directly into lesions (intra-lesion administration) in appropriate cases.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other implants, delivery systems, and modules are known to those skilled in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Therapeutic and Prophylactic Methods

The fusion proteins of this invention are useful to treat, e.g., ablate or kill, an aberrant cell, e.g., an aberrant PSMA-expressing prostate cell, or a non-malignant, nonprostatic, hyperproliferative cell. The method includes contacting the cell, or a vascular endothelial cell proximate to the cell, with a fusion protein, e.g., a fusion protein described herein, that binds specifically PSMA and delivers the endopeptidase in an amount sufficient to ablate or kill the cell.

Thus, the invention features methods of treating or preventing a disorder, e.g., a prostatic disorder (e.g., a cancerous or non-cancerous disorder, e.g., a benign or hyperplastic prostatic disorder) or a non-prostatic disorder (e.g., a cancer, e.g., a malignant cancer) by administering to a subject a fusion protein described herein, in an amount effective to treat or prevent such disorder. Examples of prostatic disorders that can be treated or prevented include, but are not limited to, genitourinary inflammation (e.g., inflammation of smooth muscle cells) as in prostatitis; benign enlargement, for example, nodular hyperplasia (benign prostatic hypertrophy or hyperplasia); and cancer, e.g., adenocarcinoma or carcinoma, of the prostate and/or testicular tumors. Methods and compositions disclosed herein are particularly useful for treating metastatic lesions associated with prostate cancer, e.g., hormone refractory prostate cancer. In some embodiments, the patient will have undergone one or more of prostatectomy, chemotherapy, or other anti-tumor therapy and the primary or sole target will be metastatic lesions, e.g., metastases in the bone marrow or lymph nodes. Examples of non-prostatic cancerous disorders include, but are not limited to, solid tumors, soft tissue tumors, and particularly metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), genitals and genitourinary tract (e.g., renal, urothelial, bladder cells), pharynx, CNS (e.g., neural or glial cells), skin (e.g., melanoma), and pancreas, as well as adenocarcinomas which include malignancies such as most colon cancers, rectal cancer, renal-cell carcinoma, liver cancer, non-small cell carcinoma of the lung, cancer of the small intestine and cancer of the esophagus. In some embodiments, the subject will have undergone one or more of surgical removal of a tissue, chemotherapy, or other anti-cancer therapy and the primary or sole target will be metastatic lesions, e.g., metastases in the bone marrow or lymph nodes.

The methods of the invention may be practiced on any subject, e.g., a mammal, a higher primate preferably on humans. As used herein, the term “subject” is intended to include human and non-human animals. Preferred human animals include a human patient having a cancer as described herein. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc.

The methods described herein can also be used in combination with other known cancer treatments, e.g., radiation, etc.

### EXAMPLES

### Example 1

### Neutral Endopeptidase-Inhibits Growth and Migration of Prostate Cancer Cells

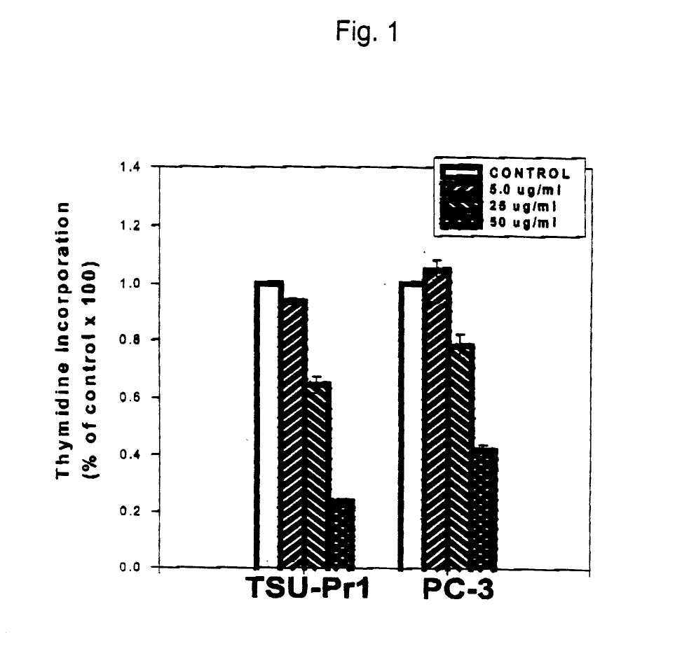

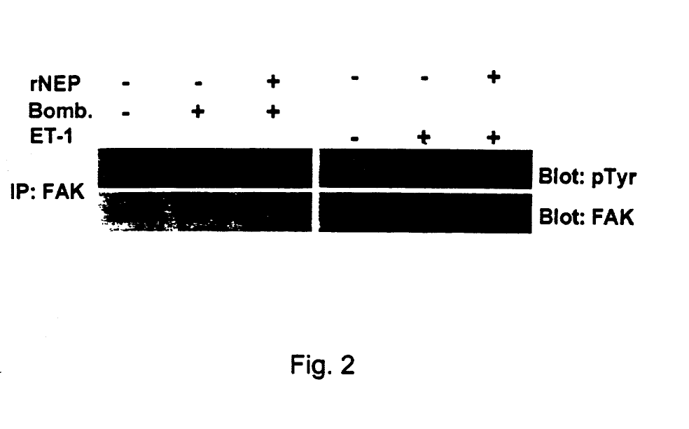

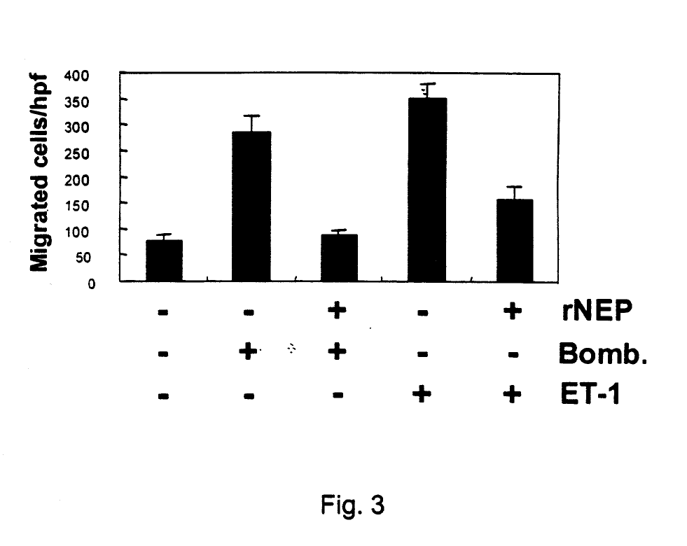

To determine the biological effects of NEP on prostate cancer cells, the effect of recombinant NEP (rNEP) on growth of the androgen-independent prostate cancer cell lines PC-3 and TSU-Pr1 was examined. Exogenous rNEP significantly inhibited thymidine incorporation in a dose dependent fashion (FIG. 1). FIGS. 2 and 3 demonstrate NEP expression, FAK phosphorylation and cell migration in prostate cancer cells. Referring to FIG. 2, TSU-Pr1 cells were cultured in media without FCS for 24 hours (Lanes 1 and 4), followed by the addition of 10 nM bombesin. (Bomb., lane 2) or 10 nM ET-1 (lane 5) for 20 minutes, or by the addition of 50 μg/ml of rNEP for 2 hours, and then bombesin (lane 3) or 10 ET-1 (lane 6) for 20 minutes. Cells were lysed and 300 μg of total cell lysates were immunoprecipitated with anti-FAK antibody C-20, separated by SDS-PAGE, transferred to nitrocellulose and Western blotted with anti-pTyr monoclonal antibody PY20. FIG. 3 shows cell migration assays performed under conditions identical to those of FIG. 2. In FIG. 3, the bars represent standard deviations. NEP neuropeptide substrates bombesin and 'ET-1 stimulated phosphorylation of FAK and promoted the migration of androgen-independent prostate cancer cells through ECM but had minimal effects on NEP-expressing LNCaP cells. Focal adhesion kinase (FAK) is believed to play a role in modulating cell migration of prostate cancer cells, and is activated through tyrosine phosphorylation on tyrosine-397, which is necessary for FAK-promoted cell migration. Inhibition of endogenous NEP enzymatic activity in LNCaP cells using the NEP competitive enzyme inhibitor CGS24592 resulted in increased FAK phosphorylation on tyrosine and a 4.3-fold increase in LNCaP migrated cell number compared with untreated control cells (p<0.005). Similar experiments in TSU-Pr1 cells cultured in media containing FCS showed rNEP can inhibit FAK phosphorylation and cell migration in a time and dose dependent fashion (data not shown). In addition, bombesin or ET-1 stimulated increase in the levels of phosphorylated FAK (22 and 26-fold increase, respectively) in TSU-Pr1 cells cultured in media without serum for 24 hours (FIG. 2, lanes 2 and 5), is significantly inhibited by pretreatment with rNEP for 2 hours (lanes 3 and 6). Bombesin and ET-1 induced cell migration of TSU-Pr1 cells (3.6-fold for bombesin, p<0.005; 4.4-fold for ET-1, p<0.005) is also blocked by rNEP (FIG. 3).

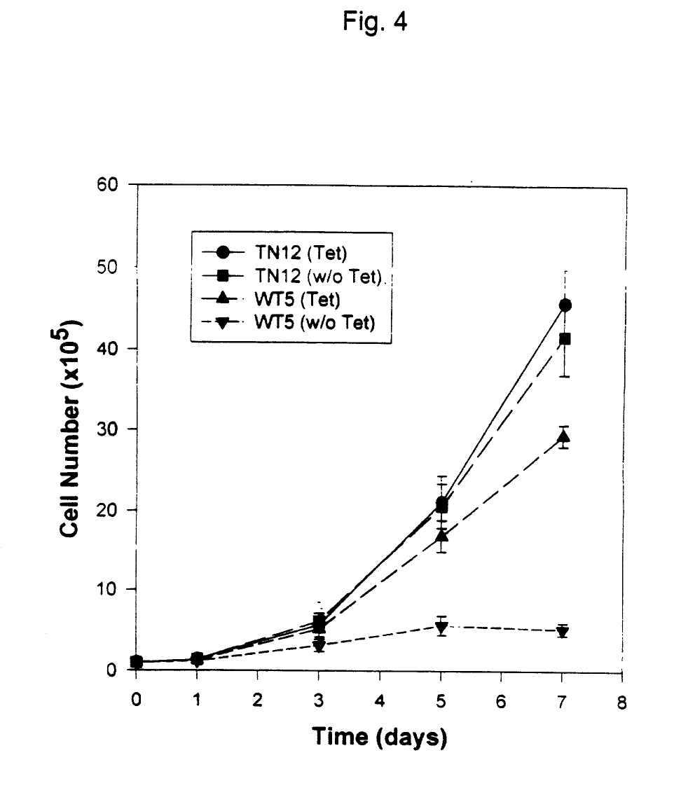

To investigate the effects of overexpressing NEP at the cell surface, an inducible tetracycline-regulatory expression system was used to introduce and express the NEP gene in TSU-Pr1 cells, generating WT-5 cells, which express high levels of enzymatically active NEP protein when cultured in the absence of tetracycline. TN12 cells, which contain the identical vectors without the NEP gene and do not express NEP, were used as the control. Expression of NEP from WT-5 cells following removal of tetracycline from the media resulted in >80%inhibition in cell proliferation over one week (p<0.005) compared to control cells (FIG. 4). Tetracycline removal resulting in NEP expression in WT-5 cells also resulted in >95% decrease in FAK phosphorylation and >90% decrease in cell migration (p<0.005), but FAK phosphorylation or cell migration in control TN-12 cells were not affected. Analysis of the mechanisms of NEP induced growth suppression revealed that NEP induced G1 cell cycle arrest, a four-fold increase in the number of prostate cancer cells undergoing apoptosis, and an increase in the level of unphosphorylated retinoblastoma protein. NEP effects on cell migration result from NEP induced inhibition of FAK association with cSrc, which is required for neuropeptide-mediated FAK phosphorylation and cell migration.

### Example 2

### NEP Expression Inhibits Tumor Growth in Athymic Mice

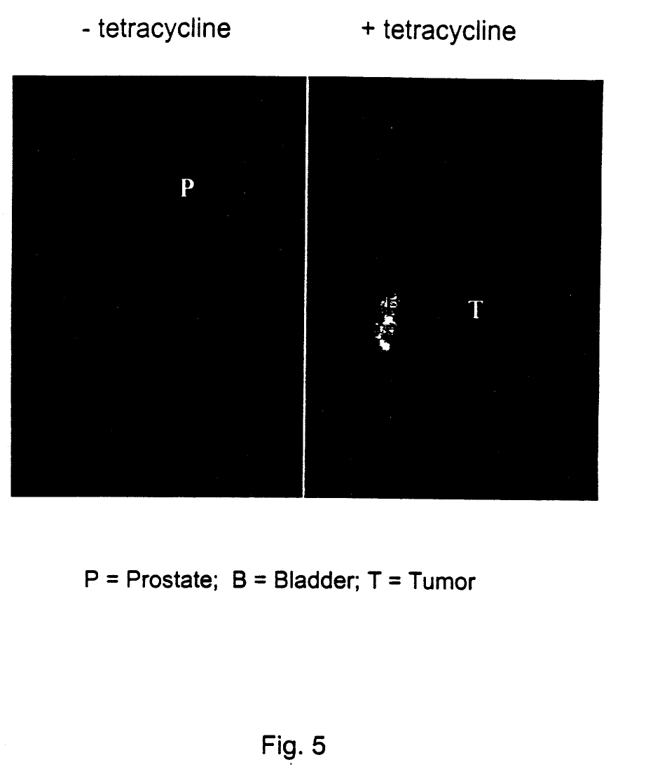

To determine if NEP could inhibit tumorigenicity in vivo, recombinant NEP was administered intraperitoneally for 30 days to athymic mice injected in the flank with TSU-Pr1 cells. However, significant inhibition of tumor growth was not observed. This is not surprising, since sustained high levels of rNEP in serum are difficult to maintain. The effect of expressing cell-surface NEP was, therefore, examined. WT5 and TN12 cells were injected directly into the prostate gland of athymic mice. One half of the animals received doxycyiine in their feed, and all animals were sacrificed at 30 days. Magnetic resonance imaging was performed on one animal from each treatment group prior to sacrifice. Tumors were detected in the prostate of two animals injected with TN12 cells regardless of whether they received tetracycline (not shown), and in the prostate of one animal fed with tetracycline (NEP expression oppressed) that was injected with WT5 cells (FIG. 5, right image). However, no tumor was detected in the animal injected with WT5 cells which did not receive tetracycline (NEP expressed)(FIG. 5, left image). Autopsies of all animals revealed 100% tumor formation in animals receiving TN12 cells, and in 4 of 5 (80%) animals injected with WT5 cells and fed with tetracycline. Only 1 of 5 animals injected with WT5 cells which did not receive tetracycline developed a tumor, which was appreciably smaller than other tumors formed.

### Example 3

### Construction of an NEP/Anti-PSMA Antibody Fusion Protein

Our experiments suggest that NEP has potential as therapy for androgen-independent prostate cancer. Numerous studies implicate the NEP neuropeptide substrates bombesin, ET-1 and neurotensin in the growth and development of hormone-refractory prostate cancer. Receptors for bombesin, ET-1 and neurotensin are expressed by androgen-independent prostate cancer cells. Receptor antagonists for these peptides can inhibit prostate cancer cell growth in prostate cancer tumor xenografts. One major drawback of these studies is that receptor antagonists can only target one neuropeptide. The advantage of NEP is that it can target numerous neuropeptides that contribute to androgen-independent prostate cancer. Nevertheless, there are numerous problems associated with the administration of rNEP to patients. A gene therapy approach similar to our in vitro strategy is also not practical as it may be difficult to target appropriate vectors to metastatic prostate cancer cells. Therefore, the strategy to target NEP to prostate cancer cells using a fusion protein was determined. A fusion protein containing the Fab fragment (antigen-binding fragment) of a monoclonal specific for prostate cancer fused to NEP was created to specifically deliver NEP to prostate cancer cells.

PSMA was chosen for the following reasons: 1) PSMA is expressed only by prostate cancer cells; 2) monoclonal antibodies which specifically recognize this antigen were available; and 3) the sequence encoding the Fc′ protein of these antibodies could be replaced with the NEP cDNA. In contrast to other highly .restricted prostate-related antigens such as prostate specific antigen (PSA), prostatic acid phosphatase (PAP) and prostate secretory protein (PSP), all of which are secretory proteins, PSMA is anchored to the cell membrane. Among reasons for significant interest in PSMA is that it is ideal for in vivo prostate-specific targeting strategies. In addition to its prostate specificity, PSMA is expressed by virtually all prostate cancer cells, and expression is further increased in higher grade cancers and metastatic disease as well as in hormone-refractory prostate cancer.

The plasmid pSFG-Pz1 (7,687 bp) contains the PSMA specific scFv derived from mAb J591, the CD8 hinge and transmembrane domains, and the T cell receptor cytoplasmic receptor cloned into a retroviral vector in the SFG vector backbone. Restriction digestion, PCR and cloning techniques were used to replace the CD8 and chain domains with the extracellular domain of human NEP (hNEP) cDNA and a 15 amino acid linker sequence. The plasmid pSFG-Pz1 was digested with the restriction endonucleases Not1 and BamHI, and the resulting products were purified on an agarose gel to obtain a fragment containing the vector DNA and the cDNA sequence of ssFv of J591 antibody (pSFG-NB). A polymerase chain reaction (PCR) was performed to obtain a PCR product containing the neutral endopeptidase gene (NEP) from the plasmid pChink. The 5′ primer contained a Not1 restriction site and a linker region, and the 3′ primer contained a BamHI site. The resulting PCR product was digested with NotI and BamHI and gel purified, then ligated into pSFG-NB. After transformation, the clones were screened by NotI and BamHI digestion, and the positive clones were confirmed by sequencing. One clone containing the linker sequence encoded by the 5′ primer was selected and designated pJNEP. This clone was purified and transfected into H29 cells to produce retrovirus containing hNEP cDNA, and then further infected into CHO cells. Infected CHO cells then were screened by measurement of NEP activity to select those expressing high levels of NEP.

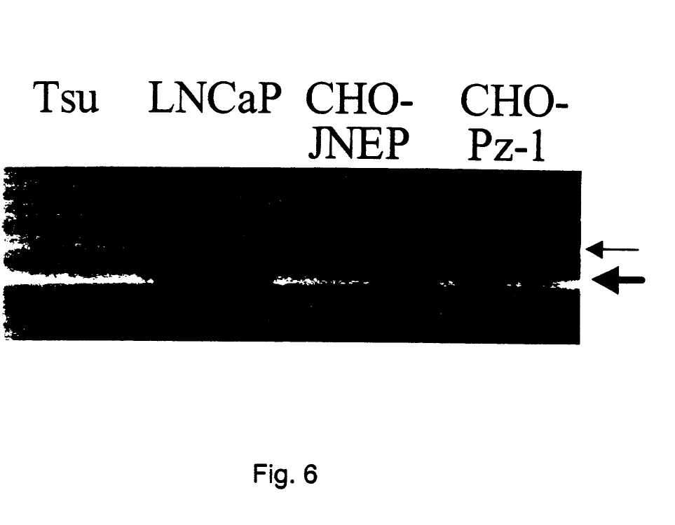

To create a fusion NEP-J591 protein, restriction digestion, PCR and cloning techniques were used to replace the CD8 and chain domains with the extracellular domain of human NEP (hNEP) cDNA and a 15 amino acid linker sequence. Selected clones were sequenced and a clone containing the correct sequence selected and labeled pJNEP. Purified retroviral stock was used to infect both NIH-3T3 and CHO cells. The Pz-1 containing retrovirus was used as control. Measurement of NEP-specific enzyme activity in supernatant and cell lysates confirmed, that CHO-JNEP and 3T3-JNEP both expressed enzymatically active NEP. Western analysis confirmed expression of the NEP-J591 fusion protein (FIG. 6). Cell lysates from CHO cells infected with JNEP or Pz-1 were separated on a 10% SDS-PAGE, transferred to nitrocellulose and blotted with anti-NEP mAb. Native NEP is roughly 110 kD while molecular weight of the fusion protein calculated to be 128.9 kD (thin arrow). Lysates from TSU-Pr1 and LNCaP (thick arrow) were used as negative control and positive controls for NEP.

### Example 4

### NEP-J591 Fusion Protein Specifically Targets PSMA-Expressing Prostate Cancer Cells

First, PC3/PSMA cells (which stably express the PSMA gene) and PC3/FLU cells (which contain the identical vector without the PSMA gene) were incubated in purified supernatant derived from CHO-JNEP and CHO-PZ1 cells overnight, washed with PBS, and NEP enzyme activity in cell lysates was measured. NEP activity was detected in cell lysates of PC-3/PSMA incubated in medium from CHO-J591 (36.7 pmol/μg protein/minute) but not in PC-3/PSMA incubated in medium from CHO-Pz1 (0.9 pmol/1 g protein/minute), or in PC-3/ FLU cells incubated with medium from either CHO-J591 (2.7 pmol/μg protein/minute) or CHO-Pz1 (1.7 pmol/μg protein/minute). Next, PC-3/PSMA and PC3/FLU cells were incubated with equal amounts of either partially purified NEP-J591 or native mAb J591, or first with mAb J591 followed by NEP-J591, to determine if pre-incubation with mAb J591 would inhibit NEP-J591 binding to PSMA, and to show that binding to PSMA by a monoclonal antibody does not increase NEP enzyme activity. Once again, there was no significant NEP-specific enzyme activity in PC-3/FLU cells incubated in NEP-J591, or PC-in 3/PSMA cells incubated in mAb J591. However, NEP-specific enzyme activity was 46 pmol/μg protein/minute in PC3/PSMA cells incubated with NEP-J591, which decreased to 20.4 pmol/μg protein/minute if the cells were first incubated for 1 hour in media containing mAb J591. Finally, immunoflourescence staining of PC3/PSMA and PC-3iFLU cells were performed using mAb muJ591, and anti-NEP mAb J5 following culturing with NEP-J591 fusion protein. Nuclear staining was also performed using PI (propidium iodide). No staining could be detected in PC-3/FLU cells (which lack PSMA expression) with either antibody (not shown). However, immunostaining was observed in PC-3/PSMA cells with both mAb J591 and NEP-J591 fusion protein (not shown). Of note, cells incubated with NEP-J591 showed increased nuclear staining suggestive of apoptotic bodies. Taken together, these data show that the NEP-anti-PSMA antibody fusion protein specifically targets PSMA-expressing prostate cancer cells, resulting in high levels of NEP-specific enzyme activity.

### Example 5

### NEP-J591 Fusion Protein Inhibits Prostate Cancer Cell Growth in vitro

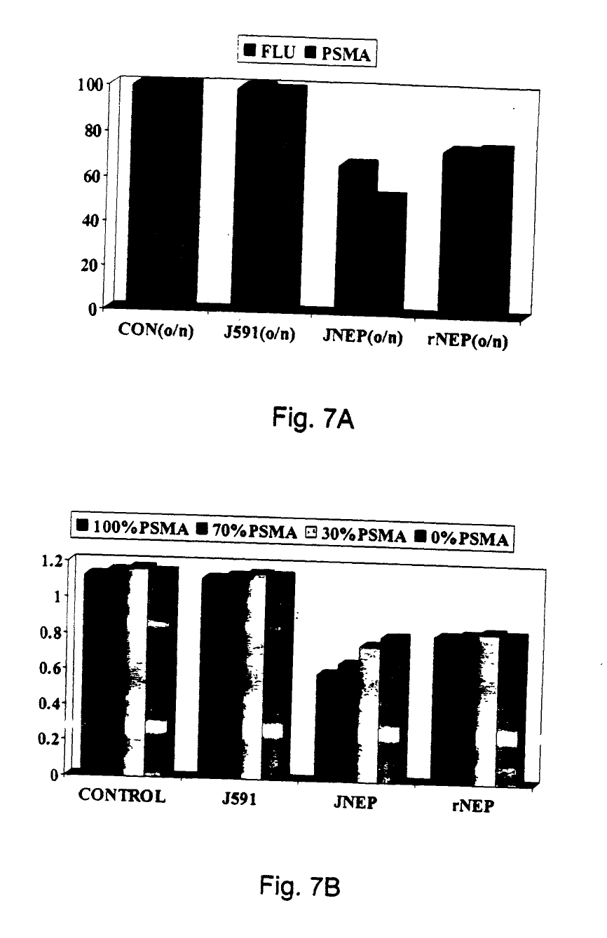

PC-3/PSMA and PC-3/FLU cells were incubated overnight in partially purified media from CHO-JNEP cells and the effect on growth determined using MTT assays (FIGS. 7A and 7B). PC-3/PSMA and PC-3/FLU cells were plated in 96 well plates, incubated with partially purified media derived from CHO-JNEP cells, mAb J591 or rNEP overnight, and an MTT assay performed the next day. Equal protein concentrations of mAb J591 and NEP-J591, and equal specific activities of NEP-J591 and rNEP were used. Recombinant NEP and mAb J591 were used as controls. Referring to FIG. 7A, NEP (rNEP and NEP-J591) inhibited growth in both cell lines, although inhibition was greatest in PC3/PSMA cells treated with NEP-J591. To confirm an increased and specific growth inhibitory effect of NEP-J591 compared to rNEP in the media, PC-3/PSMA and PC-3/FLU cells were cultures at various ratios and treated them with NEP-J591. As shown in FIG. 7B, the greatest amount of growth inhibition was observed in cultures containing 100% PC-3/PSMA cells.

Various patents and publications are cited herein, and their disclosures are hereby incorporated by reference in their entireties. The present invention is not intended to be limited in scope by the specific embodiments described herein. Although the present invention has been described in detail for the purpose of illustration, various modifications of the invention as disclosed, in addition to those described herein, will become apparent to those of skill in the art from the foregoing description. Such modifications are intended to be encompassed within the scope of the present claims.

What is claimed is:

1. A purified fusion protein comprising an endopeptidase bound to a monoclonal antibody, or an antigen binding portion thereof, specific for prostate specific membrane antigen (PSMA).

2. The purified fusion protein of claim 1, wherein said endopeptidase is a neutral endopeptidase.

3. The purified fusion protein of claim 2, wherein said neutral endopeptidase is neutral endopeptidase EC24.1.

4. The purified fusion protein of claim 1, wherein said endopeptidase cleaves one or more of atrial natriuretic factor, substance P, bradykinin, oxytocin, Leu-enkephalins, Met-enkephalins, neurotensin, bombesin, endothelin-1, and bombesin-like peptides.

5. The purified fusion protein of claim 1, wherein said antigen binding portion thereof is a member selected from the group consisting of F(ab), F(ab′)₂, scFv, and Fv.

6. The purified fusion protein of claim 5, wherein said antigen binding portion thereof is an scFv.

7. The purified fusion protein of claim 1, wherein said monoclonal antibody, or an antigen binding portion thereof, binds to an extracellular domain of PSMA.

8. The purified fusion protein of claim 1, wherein said monoclonal antibody, or an antigen binding portion thereof, is a monoclonal antibody selected from the group consisting of J591, J533, E99 and J415.

9. The purified fusion protein of claim 8, wherein said monoclonal antibody, or an antigen binding portion thereof, is monoclonal antibody J591.

10. The purified fusion protein of claim 9 , wherein said monoclonal antibody, or an antigen binding portion thereof, is an scFv antigen binding portion of monoclonal antibody J591.

11. The purified fusion protein of claim 8, wherein said monoclonal antibody, or an antigen binding portion thereof, is monoclonal antibody J533.

12. The purified fusion protein of claim 8, wherein said monoclonal antibody, or an antigen binding portion thereof, is monoclonal antibody E99.

13. The purified fusion protein of claim 8, wherein said monoclonal antibody, or an antigen binding portion thereof, is monoclonal antibody J415.

14. The purified fusion protein of claim 1, wherein said monoclonal antibody, or an antigen binding portion thereof, is a monoclonal antibody produced by a hybridoma having an ATCC Accession Number selected from the group consisting of HB-12101, HB-12109, HB-12127, and HB-12126.

15. A composition comprising: a purified fusion protein comprising an endopeptidase bound to a monoclonal antibody, or a antigen binding portion thereof, specific for PSMA; and a pharmaceutically acceptable carrier, excipient, or stabilizer.

16. A cell line that produces a purified fusion protein comprising an endopeptidase bound to a monoclonal antibody, or an antigen binding portion thereof, specific for prostate specific membrane antigen (PSMA).

17. A method for binding a purified fusion protein comprising an endopeptidase to a cell expressing PSMA on its extracellular surface, comprising:
providing a purified fusion protein comprising an endopeptidase bound to a monoclonal antibody, or an antigen binding portion thereof, specific for prostate specific membrane antigen (PSMA); and
contacting said cell with said fusion protein under conditions effective to permit binding of said fusion protein to said PSMA.

18. The method of claim 17, wherein said endopeptidase is a neutral endopeptidase.

19. The method of claim 18, wherein said neutral endopeptidase is neutral endopeptidase EC24.11.

20. The method of claim 17, wherein said endopeptidase cleaves one or more of atrial natriuretic factor, substance P, bradykinin, oxytocin, Leu-enkephalins, Met-enkephalins, neurotensin, bombesin, endothelin-1, and bombesin-like peptides.

21. The method of claim 17, wherein said antigen binding portion thereof is a member selected from the group consisting of F(ab), F(ab′)₂, scFv, and Fv.

22. The method of claim 21, wherein said antigen binding portion thereof is an scFv.

23. The method of claim 17, wherein said monoclonal antibody, or an antigen binding portion thereof, binds to an extracellular domain of PSMA.

24. The method of claim 17, where said monoclonal antibody, or an antigen binding portion thereof, is a monoclonal antibody selected from the group consisting of J591, J533, E99 and J415.

25. The method of claim 24, wherein said monoclonal antibody, or an antigen binding portion thereof, is monoclonal antibody J591.

26. The method of claim 25, wherein said monoclonal antibody, or an antigen binding portion thereof, is an scFv antigen binding portion of monoclonal antibody J591.

27. The method of claim 24, wherein said monoclonal antibody, or an antigen binding portion thereof, is monoclonal antibody J533.

28. The method of claim 24, wherein said monoclonal antibody, or an antigen binding portion thereof, is monoclonal antibody E99.

29. The method of claim 24, wherein said monoclonal antibody, or an antigen binding portion thereof, is monoclonal antibody J415.

30. The method of claim 17, wherein said monoclonal antibody, or an antigen binding portion thereof, is a monoclonal antibody produced by a hybridoma having an ATCC Accession Number selected from the group consisting of HB-12101, HB-12109, HB-12127, and HB-12126.
